(19) United States
(12) Patent Application Publication
(10) Pub. No.: US 2007/0135817 A1
(43) Pub. Date: Jun. 14, 2007

(54) Percutaneous screw assembly

(76) Inventor: Michael D. Ensign, Salt Lake City, UT (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 11/636,127

(22) Filed: Dec. 8, 2006

Related U.S. Application Data
(60) Provisional application No. 60/748,942, filed on Dec. 8, 2005.

(51) Int. Cl. A61B 17/60; A61F 2/00; A61B 17/58

(52) U.S. Cl. 606/96

(57) ABSTRACT

In one of many possible embodiments, the present exemplary system provides a connection member for coupling to one or more orthopedic fasteners that includes a fastener head securing member including a fastener head securing cavity having an axis defined by a wall member, a compressible seating member disposed within the head securing cavity, an compressible seating member coupled to a surface of the wall member, a rod coupled to the wall member, the rod being substantially transverse to the axis, and a fastener head receiving orifice formed coaxially with the axis.

### RELATED APPLICATIONS

This application claims the benefit under 35 U.S.C. § 119(e) of U.S. Provisional Patent Application No. 60/748,942 filed Dec. 8, 2005, titled “Percutaneous Screw Assembly,” which provisional application is incorporated herein by reference in its entirety.

### TECHNICAL FIELD

The present exemplary system and method relates to medical devices. More particularly, the present exemplary system and method relates to snap-on orthopedic rod placement devices.

### BACKGROUND

The use of bone stabilization/fixation devices to align or position bones is well established. Furthermore, the use of spinal bone stabilization/fixation devices to align or position specific vertebrae or a region of the spine is well established. Typically such devices for the spine utilize a spinal fixation element, comprised of a relatively rigid member such as a plate or a rod that is used as a coupler between adjacent vertebrae. Such a spinal fixation element can effect a rigid positioning of adjacent vertebrae when attached to the pedicle portion of the vertebrae using pedicle bone anchorage screws. Once the coupled vertebrae are spatially fixed in position, procedures can be performed, healing can proceed, and/or spinal fusion may take place.

Spinal fixation elements may be introduced to stabilize the various vertebrae of the spine. Some devices for this purpose are designed to be attached directly to the spine, but the generally invasive nature of the standard paraspinal approach used to implant these devices may pose drawbacks. For example, muscle disruption and blood loss may result from standard paraspinal implantation approaches.

### SUMMARY

In one of many possible embodiments, the present exemplary system provides a connection member for percutaneously coupling to one or more orthopedic fasteners that includes a fastener head securing member including a fastener head securing cavity having an axis defined by a wall member, a compressible seating member disposed within the head securing cavity, an adjustable compression member coupled to a surface of the wall member, a rod coupled to the wall member, the rod being substantially transverse to the axis, and a fastener head receiving orifice formed coaxially with the axis.

In another exemplary embodiment a connection member for percutaneously coupling one or more orthopedic fasteners having a head portion includes a housing member defining a fastener head securing cavity having an axis defined by a wall member, the housing member including a proximal end and a distal end, the fastener head securing cavity terminating at a fastener head receiving orifice formed coaxially with the axis on the distal end. Additionally, the connection member includes a compressible seating member disposed within the head securing cavity, the compressible seating member being configured to couple the head portion of the one or more orthopedic fasteners, a rod coupled to the wall member, the rod being substantially transverse to the axis, and an adjustable translation member coupled to the proximal end of the connection member, wherein the translation member is configured to initiate a compression of the compressible seating member to secure an orientation of the connection member relative to the one or more orthopedic fasteners.

Another embodiment of the present exemplary system and method provides a method for coupling a connection member to a pedicle screw including passing a head of the orthopedic fastener through a first orifice in the connection member along a first line of motion, seating the orthopedic fastener head in the connection member along the first line of motion; and securing the connection member to the orthopedic fastener.

### BRIEF DESCRIPTION OF THE DRAWINGS

The accompanying drawings illustrate various embodiments of the present system and method and are a part of the specification. The illustrated embodiments are merely examples of the present system and method and do not limit the scope thereof.

FIG. 1 is a side view of a snap-on percutaneous screw system in an assembled state, according to one exemplary embodiment.

FIG. 2 is a partially exploded view of the snap-on percutaneous screw system prior to assembly, according to one exemplary embodiment.

FIG. 3 is a flow chart illustrating a method of securing a percutaneous connection member to a head of a pedicle screw, according to one exemplary embodiment.

FIG. 4A is a cross-sectional side view of a snap-on percutaneous screw system, according to one exemplary embodiment.

FIG. 4B is an exploded cross-sectional view of a snap-on percutaneous screw system, according to one exemplary embodiment.

FIG. 4C is a cross-sectional view of an assembled snap-on system prior to insertion of a screw head, according to one exemplary embodiment.

FIG. 5A is a cross-sectional side view of a snap-on percutaneous screw system securely coupled to the head of a pedicle screw, according to one exemplary embodiment.

FIG. 5B is an exploded cross-sectional view of a snap-on percutaneous screw system, according to one exemplary embodiment.

FIG. 5C is a cross-sectional view of an assembled snap-on system prior to insertion of a screw head, according to one exemplary embodiment.

FIG. 6A is a cross-sectional side view of a snap-on percutaneous screw system provisionally coupled to a head of a pedicle screw, according to one exemplary embodiment.

FIG. 6B is an exploded cross-sectional view of a snap-on percutaneous screw system, according to one exemplary embodiment.

FIG. 6C is a cross-sectional view of an assembled snap-on system illustrating engagement of the set screw with the split ring, according to one exemplary embodiment.

FIG. 7A is a cross-sectional side view of a snap-on percutaneous screw system, according to one exemplary embodiment.

FIG. 7B is an exploded perspective view of a snap-on percutaneous screw system, according to one exemplary embodiment.

Throughout the drawings, identical reference numbers designate similar, but not necessarily identical, elements.

### DETAILED DESCRIPTION

The present specification provides a number of exemplary connection members and methods that can be used for any number of orthopedic rod placement systems. According to the present exemplary system and method, pecutaneous screw placement is facilitated. Specifically, the present exemplary systems and methods provide for the percutaneous placement of pedicle screws, followed by easy placement of the rod and tulip assembly. As will be described in further detail below, the present exemplary connection member allows for the main tulip housing and connector rod to be snapped directly onto the head of a pedicle screw, without further rotation of the tulip housing. Further details of the present exemplary percutaneous screw assembly will be provided below.

By way of example, pedicle screw systems may be fixed in the spine in a posterior lumbar fusion process via minimally invasive surgery (MIS) techniques. The systems are inserted into the pedicles of the spine and then interconnected with rods to manipulate (e.g., correct the curvature, compress or expand, and/or structurally reinforce) at least portions of the spine. Using the MIS approach to spinal fixation and/or correction surgery has been shown to decrease a patient's recovery time and reduce the risks of follow-up surgeries.

Traditional percutaneous fixation techniques are really only percutaneous in name. That is, they still require significant paraspinous tissue damage in order to fixedly couple a connector rod between two or more tulips. This is due in part to the implants that are available to the surgeon. The present exemplary system and method allows a surgeon to place spinal screws and rods via a true percutaneous approach by providing for pivoting and orientation of the rod and tulip housing beneath the skin prior to snap-on placement of the tulip.

The ability to efficiently perform spinal fixation and/or correction surgeries using MIS techniques is enhanced by the use of pedicle screw systems provided in accordance with the present exemplary systems and methods, which systems and methods provide a number of advantages over conventional systems. For example, a pedicle screw system in accordance with one embodiment of the present exemplary system and method provides the advantage that the pedicle screw may be inserted into the bone without being pre-operatively coupled with the rod-coupling assembly (hereinafter referred to as a tulip assembly). This is advantageous because the surgeon often needs to do other inter-body work after inserting the pedicle screw, but before attaching the larger and bulkier tulip assembly. Such an advantageous pedicle screw system may be even more crucial when using MIS techniques because the inter-body spatial boundaries in which the surgeon must work may be quite limited.

The term “distraction,” when used herein and when used in a medical sense, generally relates to joint surfaces and suggests that the joint surfaces move perpendicular to one another. However when “traction” and/or “distraction” is performed, for example on spinal sections, the spinal sections may move relative to one another through a combination of distraction and gliding, and/or other degrees of freedom.

In the following description, certain specific details are set forth in order to provide a thorough understanding of various embodiments of the present percutaneous pedicle screw system. However, one skilled in the relevant art will recognize that the present exemplary system and method may be practiced without one or more of these specific details, or with other methods, components, materials, etc. In other instances, well-known structures associated with pedicle screws have not been shown or described in detail to avoid unnecessarily obscuring descriptions of the embodiments of the systems and methods.

Unless the context requires otherwise, throughout the specification and claims which follow, the word “comprise” and variations thereof, such as, “comprises” and “comprising” are to be construed in an open, inclusive sense, that is as “including, but not limited to.”

Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment. Furthermore, the particular features, structures, or characteristics may be combined in any suitable manner in one or more embodiments.

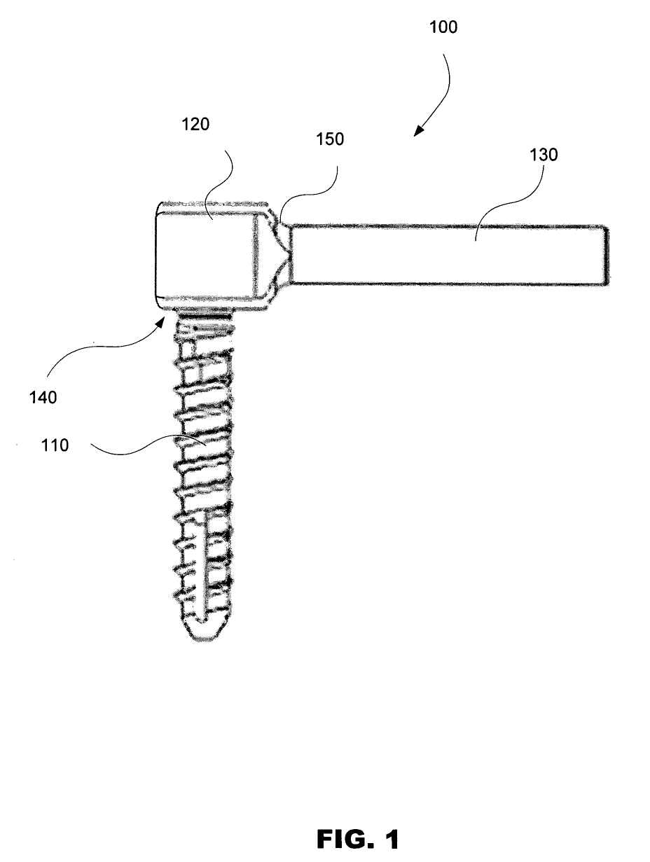

The present exemplary system and methods, illustrated by FIGS. 1 through 7B provide a connection member that can be used for any number of orthopedic rod placement systems. The connection member according to several exemplary embodiments discussed below includes a tulip and rod integrated into a single unit. Such a unit may be generally referred to as an integrated rod and tulip.

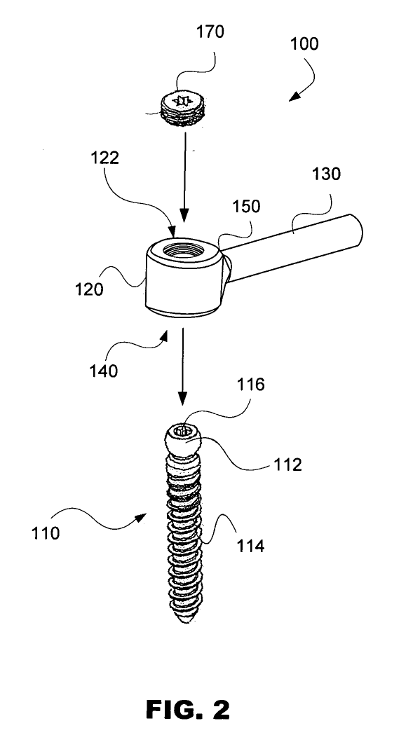

FIG. 1 illustrates a side view of a percutaneous screw assembly system (100), according to one exemplary embodiment. As illustrated in FIG. 1, the main tulip housing (120) of the screw assembly system (100) is coupled to the top portion of the pedicle screw (110). Additionally, as illustrated in FIG. 1, the connector rod (130) is directly coupled to the outer wall of the main tulip housing (120) via a rod coupling feature (150). Further, a screw head reception orifice (140) is formed in the bottom of the main tulip housing (120), allowing portions of the pedicle screw (110) to pass there through. Further details of the present exemplary percutaneous screw assembly system (100) are shown in FIG. 2.

As illustrated in FIG. 2, the exemplary pedicle screw (110) includes a pedicle screw (110) having a head or a head portion (112). According to the exemplary embodiment illustrated in FIG. 2, the pedicle screw (110) includes both an elongated, threaded portion (114) and a head portion (115). Although pedicle screws (110) are generally known in the art, the head portions (112) may be of varying configurations depending on what type of tulip assembly is to be coupled to the pedicle screw (110). The head portion (112) of the present exemplary pedicle screw (110) includes a driving feature (116) and a maximum diameter portion. The driving feature (116) of the present exemplary pedicle screw (110) permits the screw to be inserted into a pedicle bone and/or other bone. According to one exemplary embodiment, the pedicle bone is a part of a vertebra that connects the lamina with a vertebral body. Additionally, according to the present exemplary embodiment, the driving feature (116) can be used to adjust the pedicle screw (110) prior to or after the tulip assembly is coupled to the pedicle screw (110). In the illustrated embodiment, the head portion (112) of the pedicle screw (110) is coupled to the threaded portion (114) and includes a generally spherical surface with a truncated or flat top surface.

In one exemplary embodiment, the pedicle screw (110) is cannulated, which means a channel (not shown) extends axially through the entire length of the pedicle screw (110). The channel (not shown) allows the pedicle screw (110) to be maneuvered over and receive a Kirschner wire, commonly referred to as a K-wire. The K-wire is typically pre-positioned using imaging techniques, for example, fluoroscopy imaging, and then used to provide precise placement of the pedicle screw (110). While the pedicle screw (110) illustrated in FIG. 1 includes a number of components, numerous variations may be made including, but in no way limited to, varying the type of driving feature (116), varying the head shape, varying materials, varying dimensions, and the like.

In addition to the exemplary pedicle screw (110), the exemplary percutaneous pedicle screw system (100) includes a tulip assembly including a main tulip housing (120) permanently coupled to the connector rod (130) by a rod coupling feature (150). Additionally, the tulip housing (120) includes a number of features that facilitate reception, rotation, and coupling of a head portion (112) of a pedicle screw (110), according to one exemplary embodiment. As illustrated in FIG. 2, the exemplary tulip housing (120) includes a head reception orifice (140) formed on the bottom surface of the tulip housing. As shown, the tulip housing (120) includes an inner bore (122) that extends concentrically along the axis of the cylindrically shaped tulip housing, beginning at a threaded portion and exiting at the screw head reception orifice (140). According to one exemplary embodiment, the diameter of the screw head reception orifice (140) is sufficient to receive the pedicle screw head.

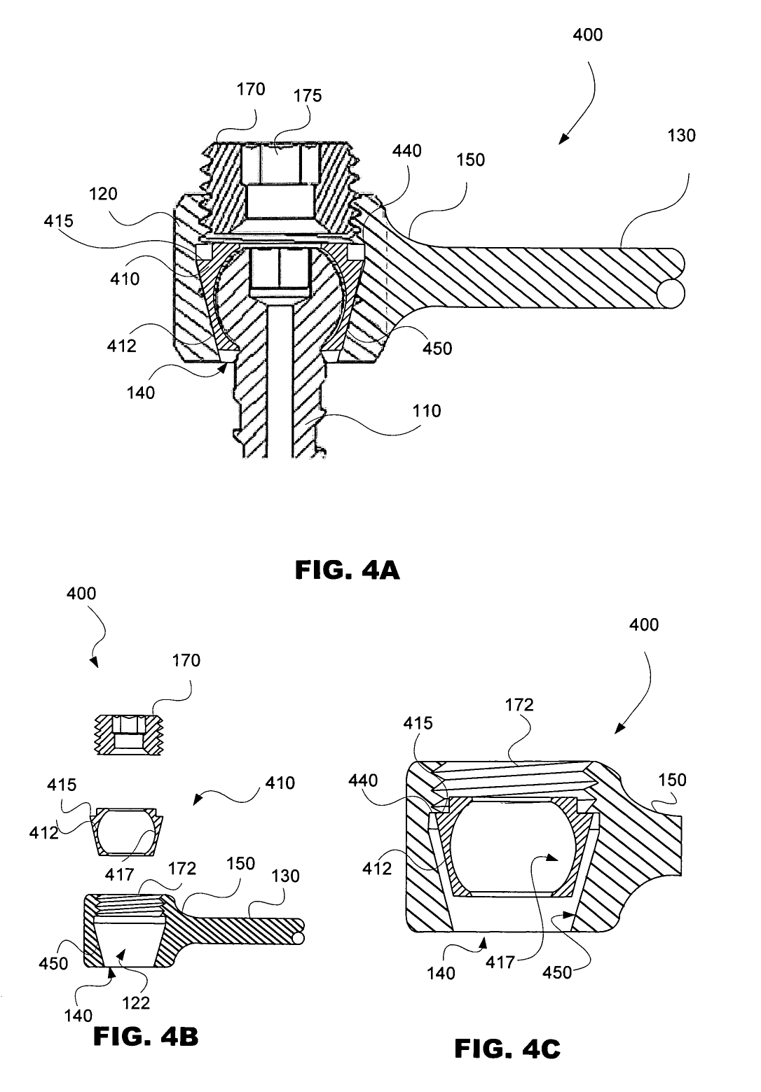

Additionally, according to one exemplary embodiment, a number of internal features and components, described below with reference to FIGS. 4A through 7B, are positioned within the inner bore (122) to receive and couple the head portion (112) of the pedicle screw (110). According to the present exemplary embodiment, the internal features and the screw head reception orifice (140) facilitate snap-on reception and provisional lock of a pedicle screw head (112). Additionally, as illustrated in FIG. 2, a set screw (170) or other fastener is inserted in the top of the inner bore (122). According to one exemplary embodiment, the set screw (170) is advanced along the threads formed on the inner bore to further engage the internal features with the pedicle screw head (112), thereby forming a secure positional lock.

As mentioned, the exemplary percutaneous pedicle screw system (100) includes the connector rod (130) securely coupled to the side wall of the tulip housing (120) by a rod coupling feature (150). According to one exemplary embodiment, the connector rod (130) may be coupled to the side wall of the tulip housing (120) using any number of joining methods known in the art including, but in no way limited to, welding, brazing, or the use of adhesives. Alternatively, the rod coupling feature (150) may include any number of mechanical joining features including, but in no way limited to, a threaded engagement feature or an interference press fit feature.

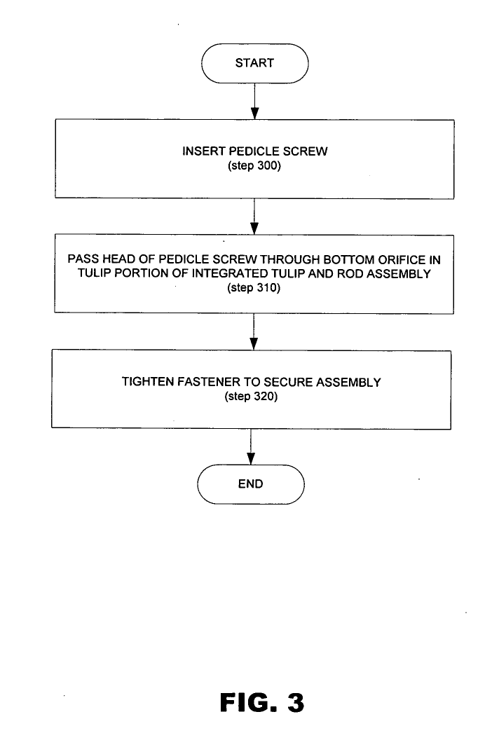

With the above-mentioned features, the present exemplary percutaneous screw assembly facilitates easy placement of the rod and tulip assembly in a desired surgical site while minimizing trauma to surrounding tissue. Particularly, as illustrated in FIG. 3, the present assembly allows for a simple insertion and coupling method. Particularly, the first step is for a surgeon to insert a pedicle screw into the desired location (step 300). As mentioned, the pedicle screw may be driven by any number of driving features into a bone mass to create an anchor. Once the pedicle screw is placed, the present exemplary percutaneous screw assembly may be introduced into the surgical site and roughly oriented.

When oriented, the head of the pedicle screw may be passed through the bottom screw head reception orifice in the tulip portion of the integrated tulip and rod assembly (step 310). With the head of the pedicle screw introduced into the tulip portion, an initial provisional engagement is established, maintaining engagement of the pedicle screw, while allowing for further movement and manipulation of the rod and tulip housing. According to this exemplary embodiment, the surgeon can perform any number of distractions or other orthopedic manipulations.

With the rod and tulip housing correctly positioned, the set screw or other fastener may be engaged to securely lock the tulip housing to the head portion of the pedicle screw (step 320). According to this exemplary embodiment, actuation of the set screw or other fastener compresses the afore-mentioned internal components about the head of the pedicle screw, positionally locking the system. Further details of exemplary internal components configured to snap onto a head portion of a pedicle screw and form a lock when engaged with a set screw or other fastener will be described in detail below with reference to FIGS. 4A through 7B.

FIGS. 4A through 4C illustrate a first exemplary snap-on percutaneous screw assembly (400), according to one exemplary embodiment. As illustrated in FIGS. 4A and 4B, the inner bore (122) of the main tulip housing (120) includes a number of internal threads on a top portion. At the termination of the internal threads is a recess, forming an annular stop (440). Continuing down the inner bore (122), the bore tapers inwardly, thereby forming a seating taper (450) that terminates at the screw head reception orifice (140). As shown, a compressible split ring (410) is disposed within the inner bore (122) of the main tulip housing (120).

According to one exemplary embodiment, the compressible split ring (410) includes a number of functional features. Particularly, as illustrated, the compressible split ring (410) includes a top protruding portion defined by an annular stop ring feature (415). From the annular stop ring feature (415), the split ring (410) tapers inwardly towards the bottom of the split ring. Additionally, as illustrated in FIG. 4B, the interior portion of the split ring (410) can include an arcuate head mating surface configured to conform to the head portion (112) of the pedicle screw (110), thereby maximizing surface contact between the two components.

Prior to insertion of the pedicle screw head, the set screw (170) is retracted from the inner bore (122) of the tulip housing (120). Consequently, the split ring (410) is allowed to advance upwardly in the inner bore (122) until the ring stop feature (415) contacts the annular stop (440), according to one exemplary embodiment. Alternatively, the set screw (170) may only be partially retracted, thereby serving as the annular stop (440). As illustrated in FIG. 4C, the upper portion of the inner bore has a larger diameter than the lower portion due to the seating taper (450). In this position, the compressible split ring (410) is allowed to expand. Consequently, when the head portion (112) of the pedicle screw (110) is advanced through the screw head reception orifice (140) and pressed against the bottom portion of the compressible split ring (410), the split ring is allowed to expand and receive the head portion (112) of the pedicle screw (110). As the head portion (112) is advanced into the head mating surface (417), the split ring compresses about the head portion (112) of the pedicle screw (110), forming a provisional lock.

When the snap-on percutaneous screw assembly is correctly oriented, the set screw (170) may be advanced to securely lock the position and orientation of the percutaneous screw assembly (400) on the head portion (112) of the pedicle screw (110). Specifically, according to one exemplary embodiment, a mating tool is inserted into the driving feature (175) of the set screw (170) and the set screw is advanced along the threaded portion of the inner bore (122). As the set screw is advanced, at least the bottom surface of the set screw (170) can contact the upper portion of the split ring (410) and/or the head portion (112) of the pedicle screw (110), thereby forcing the split ring downward in the seating taper (450). According to this exemplary embodiment, progression of the split ring (410) along the seating taper (450) compresses the compressible split ring (410) about the head portion (112) of the pedicle screw (110). When sufficiently advanced, the compressible split ring (410) imparts sufficient force on the head portion (112) of the pedicle screw (110) to positionally fix the entire percutaneous screw assembly (400) relative to the pedicle screw.

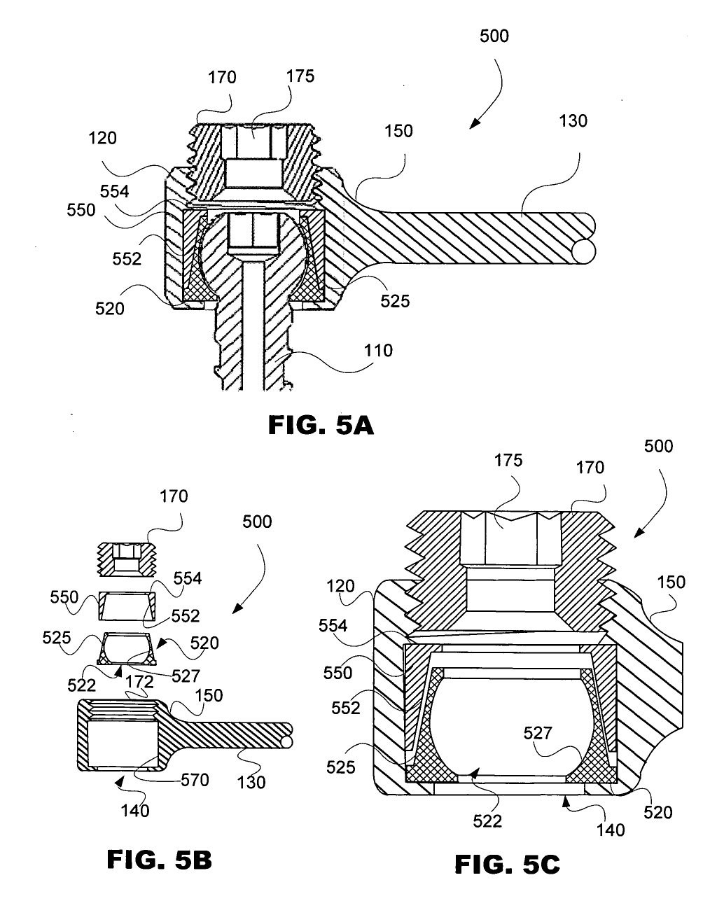

FIGS. 5A through 5C illustrate an alternative percutaneous screw assembly (500), according to one exemplary embodiment. As illustrated in FIGS. 5A and 5B, the inner housing wall (570) of the inner bore (122) is substantially parallel, rather than having a seating taper as illustrated in the previous exemplary embodiment. Rather, as shown in the present exemplary embodiment, compression is imparted to the compressible split ring (520) by a compression ring (550). Specifically, according to the exemplary embodiment illustrated in FIGS. 5A through 5C, the compression ring (550) includes a top driving surface (554) and an inner graduated surface (552). According to one exemplary embodiment, the outer surface of the compression ring (550) is substantially parallel, mirroring the inner housing wall (570).

Additionally, as illustrated in FIGS. 5A through 5C, the compressible split ring (520) includes an outer wall (525) configured to mate with the inner graduated surface (552) of the compression ring (550). Further, the compressible split ring (520) includes an inner head mating surface (527) and a head receiving orifice (522). When assembled, the graduated surface (552) is oriented over the outer wall (525) of the compressible split ring. When the set screw (170) is retracted, the compression ring (550) and the compressible split ring (520) are allowed to separate, allowing for expansion of the compressible split ring (520), as shown in FIG. 5C. This configuration allows the split ring (520) to expand and receive the head portion (112) of a pedicle screw (110). As the head portion (112) is advanced into the head mating surface (527), the split ring (520) compresses about the head portion (112) of the pedicle screw (110), forming a provisional lock.

When the snap-on percutaneous screw assembly is correctly oriented, the set screw (170) may be advanced to securely lock the position and orientation of the percutaneous screw assembly (500) on the head portion (112) of the pedicle screw (110). Specifically, according to one exemplary embodiment, a mating tool is inserted into the driving feature (175) of the set screw (170) and the set screw is advanced along the threaded portion of the inner bore (122). As the set screw is advanced, at least the bottom surface of the set screw (170) contacts the driving surface (554) of the compression ring (550), forcing the compression ring (550) into the compressible split ring (520). According to this exemplary embodiment, progression of the compression ring (550) into the split ring (520) compresses the compressible split ring (520) about the head portion (112) of the pedicle screw (110). When sufficiently advanced, the compressible split ring (520) imparts sufficient force on the head portion (112) of the pedicle screw (110) to positionally fix the entire percutaneous screw assembly (500) relative to the pedicle screw.

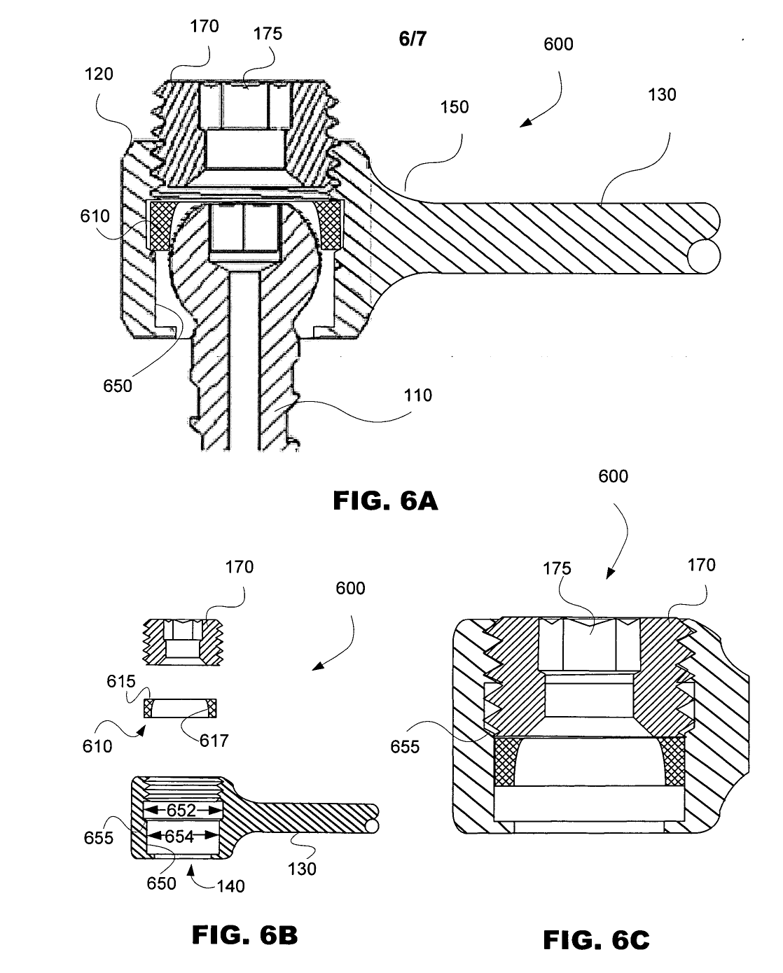

Turning now to FIGS. 6A through 6C, a third exemplary percutaneous screw assembly (600) is illustrated. As shown in FIGS. 6A through 6C, the lower portion of the inner bore (122) includes a stepped wall (650) having a first larger diameter (652) at the top portion near the threads, and a second smaller diameter (654) disposed near the screw head reception orifice (140). Additionally, as illustrated, an angled transition surface (655) may be disposed between the first large diameter (652) and the second smaller diameter (654), facilitating translation there between. Additionally, as shown in FIGS. 6A through 6C, a compressible split ring (610) is disposed within the inner bore (122). Similar to the previously described compressible split rings, the present compressible split ring (610) includes a top driving surface (615) and an inner head mating surface (617) configured to mate with the outer surface of the head portion (112) of a pedicle screw (110).

According to the present exemplary embodiment, when the compressible split ring (610) is disposed in the first large diameter portion (652) of the inner bore (122), the split ring is expanded and may receive the head portion (112) of a pedicle screw. Once the percutaneous screw assembly is correctly positioned, the set screw (170) is advanced, contacting the driving surface (615) and/or the head portion (112) of the pedicle screw (110). According to this exemplary embodiment, the compressible split ring (610) is then forced down into the second smaller diameter portion (654) of the inner bore (122). When in the second smaller diameter portion (654) of the inner bore (122), the compressible split ring (610) is compressed about the head portion (112) of the pedicle screw (110). When sufficiently advanced, the compressible split ring (610) imparts sufficient force on the head portion (112) of the pedicle screw (110) to positionally fix the entire percutaneous screw assembly (600) relative to the pedicle screw.

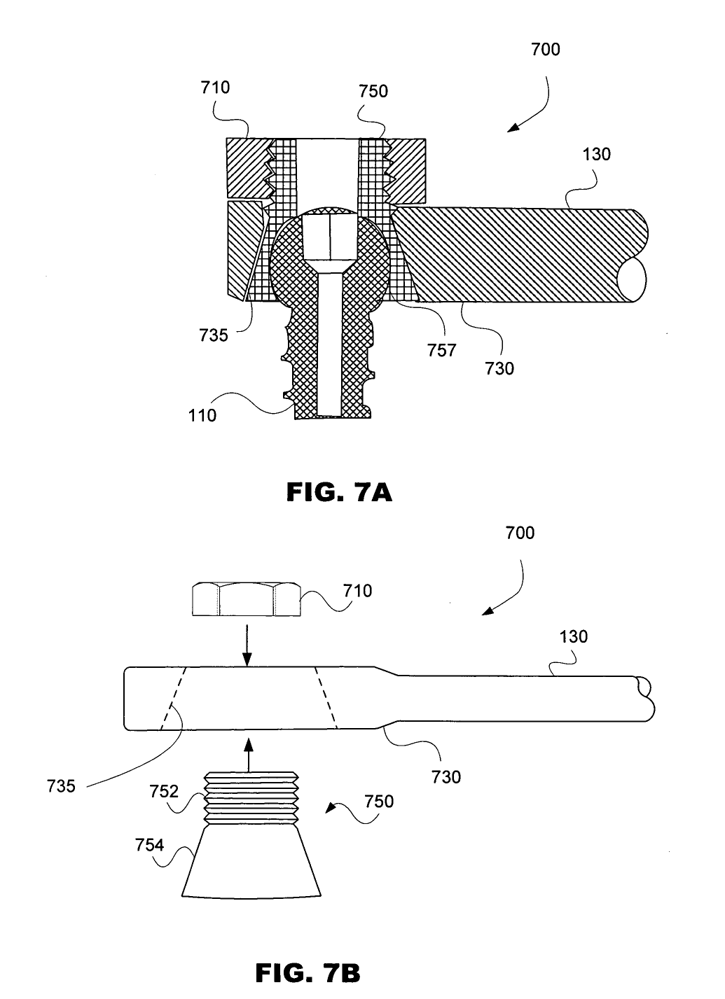

While the above-mentioned exemplary embodiments are detailed in the context of using an internal fastener in the form of a set screw, any number of fasteners may be used, including, but in no way limited to, external fasteners. Particularly, as illustrated in FIGS. 7A and 7B, a threaded split ring (750) may be used to form an exemplary percutaneous screw assembly (700), according to one exemplary embodiment. As shown in FIG. 7A, a threaded split ring (750) can include an upper threaded portion including external threads (752). Further, a lower portion of the exemplary threaded split ring (750) includes an exterior graduated surface (754) and an internal arcuate head mating surface (757).

As illustrated, a tulip member (730) including a connector rod (130) includes an internal seating taper (735) sized to pass the external threaded portion of the threaded split ring (750), while interfering with the graduated surface (754). When the threaded split ring (750) has been passed through the seating taper portion (735), a fastener such as a nut (710) may engage the external threads (752) of the split ring (750).

During operation, the fastener may either not be engaged, or only slightly engaged, thereby minimizing the interference between the seating taper (735) of the tulip member (730) and the graduated surface (754) of the threaded split ring (750). In this configuration, the threaded split ring is allowed to expand to receive the head portion (112) of a pedicle screw within the arcuate head mating surface (757). Once the percutaneous screw assembly is correctly positioned, the fastener (710) is advanced, pulling the threaded split ring (750) into the seating taper (735) of the tulip member (730). When the threaded split ring (750) is advanced in the seating taper (735), the compressible threaded split ring (750) is compressed about the head portion (112) of the pedicle screw (110). When sufficiently advanced, the compressible threaded split ring (750) imparts sufficient force on the head portion (112) of the pedicle screw (110) to positionally fix the entire percutaneous screw assembly (700) relative to the pedicle screw.

According to the present exemplary system and method, percutaneous screw placement is facilitated. Specifically, the present exemplary systems and methods provide for placement of the pedicle screws first, followed by easy placement of the integrated rod and tulip assembly. Retaining and/or locking members may be provided with the integrated rod and tulip to form an orthopedic rod placement assembly. Each assembly may be coupled to a percutaneous screw by snapping the integrated rod and tulip directly onto the head of a pedicle screw in an initial orientation. In particular, each assembly is configured to be first snapped into an initial position relative to the head of the pedicle screw. Thereafter, the retaining and/or locking members may be engaged to lock the position of the integrated rod and tulip assembly relative to the percutaneous screw.

Furthermore, due to the fixed connection between the rod and the tulip of the present exemplary system configuration, the profile and volume of the present exemplary system are reduced, compared to traditional systems.

The present exemplary system and method are elegant solutions to maintaining polyaxial movement in the orthopedic rod placement system. Additionally, according to one exemplary embodiment, the illustrated connection member may be cannulated.

A number of preferred embodiments of the present exemplary system and method have been described and are illustrated in the accompanying Figures. Nevertheless, it will be understood that various modifications may be made without departing from the spirit and scope of the present exemplary systems and methods. For example, while the exemplary implementations have been described and shown using screws to anchor into bony structures, the scope of the present exemplary system and methods is not so limited. Any means of anchoring can be used, such as a cam, screw, staple, nail, pin, or hook.

The preceding description has been presented only to illustrate and describe embodiments of invention. It is not intended to be exhaustive or to limit the invention to any precise form disclosed. Many modifications and variations are possible in light of the above teaching. It is intended that the scope of the invention be defined by the following claims.

What is claimed is:

1. A connection member for coupling to one or more orthopedic fasteners having a head portion, comprising:
a housing member defining a fastener head securing cavity having an axis defined by a wall member, said housing member including a proximal end and a distal end, said fastener head securing cavity terminating at a fastener head receiving orifice formed coaxially with said axis on said distal end;
a compressible seating member disposed within said head securing cavity, said compressible seating member being configured to couple said head portion of said one or more orthopedic fasteners;
a rod coupled to said wall member, said rod being substantially transverse to said axis; and
an adjustable translation member coupled to said proximal end of said connection member, wherein said translation member is configured to initiate a compression of said compressible seating member to secure an orientation of said connection member relative to said one or more orthopedic fasteners.

2. The connection member of claim 1, wherein said adjustable translation member comprises a set screw.

3. The connection member of claim 1, wherein said adjustable translation member comprises a nut.

4. The connection member of claim 1, wherein said compressible seating member comprises a split ring.

5. The connection member of claim 1, wherein said compressible seating member is configured to:
expand to facilitate receipt of said fastener head through said fastener head receiving orifice; and
selectively contract in response to translation of said adjustable translation member to secure said fastener head in said fastener head securing cavity.

6. The connection member of claim 4, wherein said adjustable translation member is configured to be selectively translated to compressibly fix said connection member relative to said fastener head.

7. The connection member of claim 1, wherein said compressible seating member disposed within said head securing cavity comprises:
a split ring including an inner head mating surface and an outer wall; and
a compression ring including a substantially parallel outer wall and a graduated inner wall;
wherein said graduated inner wall is configured to receive said outer wall of said split ring and selectively compress said split ring.

8. The connection member of claim 1, wherein said fastener head securing cavity comprises a tapered surface leading to said fastener head receiving orifice;
Said tapered surface being configured to compress said compressible seating member.

9. The connection member of claim 1, wherein said fastener head securing cavity is defined by a first upper diameter and a second lower diameter;
wherein said first upper diameter is larger than said second lower diameter;
said first upper diameter being configured to permit expansion of said compressible seating member; and
said second lower diameter being configured to compress said compressible seating member.

10. The connection member of claim 9, further comprising an angled transition surface between said first upper diameter and said second lower diameter.

11. The connection member of claim 1, wherein said compressible seating member is configured to receive said head portion and establish a provisional lock with said head portion prior to a compression of said compressible seating member.

12. The connection member of claim 1, wherein said compressible seating member comprises a split ring including external threads;
wherein said split ring is sized to extend beyond said head securing cavity when engaged with said adjustable translation member.

13. A connection member for coupling to one or more orthopedic fasteners having a head portion, comprising:
a housing member defining a fastener head securing cavity having an axis defined by a wall member, said cavity having a proximal end and a distal end, and said fastener head securing cavity terminating at a fastener head receiving orifice formed coaxially with said axis on said distal end of said head securing cavity;
a compressible seating member disposed within said head securing cavity, said compressible seating member being configured to couple said head portion of said one or more orthopedic fasteners;
a rod coupled to said wall member, said rod being substantially transverse to said axis; and
a set screw coupled to a proximal end of said connection member, wherein said set screw is configured to compress said compressible seating member to secure an orientation of said connection member relative to said one or more orthopedic fasteners.

14. The connection member of claim 13, wherein said compressible seating member disposed within said head securing cavity comprises:
a split ring including an inner head mating surface and an outer wall; and
a compression ring including a substantially parallel outer wall and a graduated inner wall;
wherein said graduated inner wall is configured to receive said outer wall of said split ring and selectively compress said split ring.

15. The connection member of claim 13, wherein said fastener head securing cavity comprises a tapered surface leading to said fastener head receiving orifice;
Said tapered surface being configured to compress said compressible seating member.

16. The connection member of claim 13, wherein said fastener head securing cavity is defined by a first upper diameter and a second lower diameter;
wherein said first upper diameter is larger than said second lower diameter;
said first upper diameter being configured to permit expansion of said compressible seating member; and
said second lower diameter being configured to compress said compressible seating member.

17. A method for coupling a connection member including a tulip formed to a rod to at least one orthopedic fastener having a fastening shaft comprising:
passing a head of said orthopedic fastener through a first orifice in said connection member along a first line of motion;
seating said orthopedic fastener head in said connection member along said first line of motion; and
securing said connection member to said orthopedic fastener.

18. The method of claim 17, wherein said coupling of said connection member to said at least one orthopedic fastener is performed.

19. The method of claim 18, wherein said passing a head of said orthopedic fastener through a first orifice comprises passing said connection member through a tube tulip first.

20. The method of claim 18, wherein said passing a head of said orthopedic fastener through a first orifice comprises passing said connection member through a tube rod first.

21. The method of claim 17, wherein said securing said connection member to said orthopedic fastener comprises engaging a set screw in said connection member.
